(19) United States
(12) Patent Application Publication
(10) Pub. No.: US 2021/0282943 A1
(43) Pub. Date: Sep. 16, 2021

(54) APPARATUS FOR PROSTHETIC LEG VACUUM UNIT BATTERY RECHARGING

(71) Applicants: Alex C. Magnuson, La Crosse, WI (US); Avery S. Schams, La Crosse, WI (US); Thaying Vang, Onalaska, WI (US); Tanner J. Boge, La Crosse, WI (US); Thomas R. Kujak, La Crosse, WI (US); Caitlin A. Coffey, La Crosse, WI (US); Seth C. Petersen, La Crosse, WI (US); Austin M. Sather, La Crosse, WI (US); Paige M. Treakle, La Crosse, WI (US); David M. Foye, La Crosse, WI (US); Steven G. Johnston, Genoa, WI (US)

(72) Inventors: Alex C. Magnuson, La Crosse, WI (US); Avery S. Schams, La Crosse, WI (US); Thaying Vang, Onalaska, WI (US); Tanner J. Boge, La Crosse, WI (US); Thomas R. Kujak, La Crosse, WI (US); Caitlin A. Coffey, La Crosse, WI (US); Seth C. Petersen, La Crosse, WI (US); Austin M. Sather, La Crosse, WI (US); Paige M. Treakle, La Crosse, WI (US); David M. Foye, La Crosse, WI (US); Steven G. Johnston, Genoa, WI (US)

(21) Appl. No.: 16/815,548

(22) Filed: Mar. 11, 2020

(51) Int. Cl. A61F 2/70; H02J 7/34; H02J 7/00; H02J 7/14

(52) U.S. Cl. CPC A61F 2/70; H02J 7/345; H02J 7/007; A61F 2002/6642; H02J 7/0047; H02J 7/143; H02J 2207/20; H02J 7/00032

(57) ABSTRACT

A mechanism to harvest the mechanical energy available during prosthetic leg use for the purpose of charging an electric battery operating a vacuum pump for a prosthetic leg residual limb mounting socket is disclosed. The present invention is intended to be applied with variations of leg prostheses equipped with prosthetic feet constructed with flexible heel and/or forefoot members in the sole. The present invention utilizes one or more electric generators to convert the mechanical energy occurring as bidirectional movement in the foot sole flexible members to electrical energy. The electrical energy is harvested by storage in capacitors, then subsequently used to charge the vacuum pump battery. The objective is to extend the useful operating time of the vacuum pump supporting vacuum socket mounting of the residual limb.

### FIELD

The present invention relates to the harvesting of mechanical energy from a prosthetic foot equipped prosthetic leg during ambulation for the purpose of providing electrical energy to operate a prosthetic leg vacuum pump used for vacuum socket mounting of the residual limb. The harvested energy provides internal battery recharging for continued vacuum pump operation improving support for variations in user ambulatory activity, and reducing requirements for external battery recharging equipment.

### BACKGROUND

Prosthetic legs have been needed throughout recorded history by people to maintain physical activity and address daily life needs after leg amputation. Advances in medical, mechanical, and electronics technologies applied to prosthetic inventions have enabled improvements in prosthetic leg performance, comfort, and health effects for users.

Prosthetic leg and foot variations in users include those with above knee, knee, below knee, and ankle amputations. A method for the mounting of a prosthesis to complete an amputee's working leg and foot has been to equip the prosthetic leg with a socket to receive the residual limb.

In the last 25 years, a technical improvement has been the use of a vacuum assisted (also referred to as hypobaric) socket. This has been found to improve the fit of the residual limb to the socket, which improves comfort and avoids tissue damage in use. It also has been found to improve residual limb blood circulation and tissue fluid levels, improving user health. Due to the large variation in residual limb mounting needs and user age, the vacuum assisted socket mounting method has been found to be suitable for a large number, but not all, amputees.

A drawback of the vacuum assisted socket mounting method has been the need for a source of controlled vacuum to the socket. Another drawback of the vacuum assisted socket mounting method is that the vacuum must be maintained to provide retentive force of the prosthetic to the residual limb during active use by the wearer. This has resulted in a variety of inventions using mechanical or electrical pumps to provide the necessary pressure control and evacuation of the socket.

For the methods and devices using electrically operated vacuum pumps, a battery is generally employed to provide operating power, which needs to be periodically replaced or recharged. A variety of technologies have been identified to address this need, including inductive coupling, radio frequency broadcast, direct wire connection, fluid turbine/generator combinations, and others.

A variety of inventions have been devised to harvest electrical energy from human leg motion which could be used to accomplish vacuum pump battery recharging. Ambulation causes forces and flexing across all elements of the foot and leg, with typical force durations, timing, and available energy for harvesting varying significantly. Accordingly, energy harvesting techniques used at each location vary significantly, and the availability of energy for harvesting varies by the form of limb use in ambulation. All variations of leg amputations have the prosthetic foot element in common as an available mounting location and energy harvesting source. Access to this energy can be accomplished with minimal intrusion of the prosthetic foot by using the force and movement accessible at the foot sole. Ambulation creates periodic but intermittent, discontinuous, and often relatively high values of force and movement in the foot sole which occur over a relatively short period of time. Because of the relatively short duration of high force values applied to the foot during ambulation, particularly at the heel, the form of each force and time function event is often referred to as a pulse or impulse.

A number of inventions accomplish energy harvesting at the foot sole, using various methods to convert impulsive energy to the required continuously available energy. Although these inventions are capable of harvesting energy for prosthetic equipment use, they do not meet a number of other physical requirements for many prosthetic leg users employing electrically operated vacuum pumps. An additional complication in prosthetic leg application is that although adequate mechanical energy is available for harvesting, the distribution and form of energy presented to the prosthetic varies significantly by user activity level, design of prosthetic device being used, and the specific amputation form and response of the user.

With an increasing population of prosthetic leg users, and improvements in health care resulting in increased longevity of prosthetic leg users, an overall increased demand for prosthetic leg devices which improve quality of life continues to develop. Improvements in the overall cost and performance of prosthetics is a societal challenge. Key problems in cost and quality of prosthetic devices are addressed as they are experienced by users and identified by technologists.

A problem known to be in the art with battery powered vacuum pumps is that they need either battery replacement or some means to accomplish battery recharging often on a daily basis, causing the user to experience reliability problems in the vacuum assisted socket mounting method or inconvenience in maintaining it.

Another problem known to be in the art is that harvesting energy from the impact and flexing of the foot sole requires effective response to and energy conversion of the short duration bidirectional impulsive forces which are developed in use of the prosthetic foot.

Another problem known to be in the art is that prosthetic feet are produced and applied in a variety of flexible sole element configurations to meet the needs and preferences of the user. The present variations in heel application forms include single and dual heel elements. The present variations in forefoot application forms include single, dual, and triplet forefoot elements.

Another problem known to be in the art is that a designed mechanism to harvest bidirectional force impulses often results in additional mechanical mechanisms which convert the bidirectional force impulses into stored energy and more continuous mechanical rotation to operate electric generators. The additional mechanical components add weight and mounting volume which affect the application aesthetics, user felt perceptions, and subsequently the user control abilities of the customized prosthetic.

Another problem known to be in the art is that many available technologies used to harvest, store, and control electrical energy cause additions to prosthetic component complexity and weight, adding restrictions and additional use requirements to the user with subsequent reductions in convenience and reliability.

Another problem known to be in the art is that the demanding process of accomplishing acceptable fit, form, and function for the amputee by the prosthetic clinician is complicated by the presence of additional equipment and may not be possible without reducing equipment additions to prosthetic weight, volume, and physical profile.

Another problem known to be in the art is that new prosthetic designs have improved prosthetic foot performance by dividing the physical foot into multiple support elements, more closely emulating a natural foot form. User gait patterns and support element forces are significantly affected, creating challenges to energy harvesting not addressed by many existing solutions.

Another problem known to be in the art is that existing designs for the various components used in the inherently customized prosthetic leg would require significant prosthetic component redesign or inherent incorporation in the prosthetic design.

Another problem known to be in the art is that by requiring existing design modification to an inherently customized prosthetic the addition of an optional battery charging function may not be economically feasible for an individual user.

Another problem known to be in the art is the need for external charging equipment and the need for proximity to that external charging equipment to accomplish battery recharging, which adds use restrictions and inconvenience to the user.

Another problem known to be in the art is that the recharging process often requires that the prosthetic and vacuum pump not be in use during the recharging process, adding further use restrictions and inconvenience to the user.

Another problem known to be in the art is that the status of the battery and the need for recharging or replacement may not be determined by the user without the use of a unique external device or connection.

### SUMMARY

An assembly of mechanical, electrical, and electronic components in a prosthetic foot harvests mechanical energy from the prosthetic foot sole during ambulation, stores it as electrical energy, and manages delivered energy to prosthetic leg equipment. Electrical energy is delivered to recharge the battery supplying electrical energy to a vacuum pump for a prosthetic leg residual limb mounting socket, while minimizing prosthetic equipment effects and deviations from desired form and weight. In addition, operating electrical energy is delivered to the vacuum pump while in use, as well as reporting battery status using an RF wireless link to an external user device.

It is a primary object and intent of the present invention to extend the operating service life of a battery operated vacuum assisted socket mount without requiring battery recharging using an external energy source, thereby improving the reliability to the user. Reliability is improved by extending the service time of the prosthetic leg vacuum socket residual limb mount by the addition of energy recovered from the activity of ambulation to the available vacuum pump battery energy. Reliable operation of the vacuum assisted socket mount is a priority to the user as the loss of socket mounting force results in a loss of prosthetic function, and inconvenience to the user.

It is an object and intent of the present invention to provide a mechanical and electric generator mechanism that can harvest energy from and be responsive to bidirectional foot sole force impulses and movement during normal ambulation. The bidirectional forces presented to a foot sole are a technical challenge to successful energy harvesting due to their short time duration and impulsive form.

It is an object and intent of the present invention to easily adapt to variations in prosthetic foot construction and sole element design by minimizing the number of mounting points and using common available space above the sole and below the ankle found in typical flexible prosthetic foot forms.

It is an object and intent of the present invention to achieve a mechanical and electric generator mechanism that can harvest, store, and use the available bidirectional impulsive foot sole energy and have minimal impact on the prosthetic leg's physical and mechanical design element forces, weight, and feel. By keeping the weight and force impact on the prosthesis in application as perceived by the user small, and minimizing the energy harvesting force impact on prosthetic operation, control adaption of the prosthetic by the user is improved.

It is an object and intent of the present invention to achieve a mechanical and electric generator mechanism and control means that can harvest the bidirectional impulsive foot sole energy, provide temporary energy storage, regulate operating voltage, and control battery charging while the vacuum pump is operating. Providing these functions reliably delivers improved prosthetic vacuum socket mount utility, aiding the health and convenience to the user.

It is an object and intent of the present invention to support the harvesting of energy from multiple prosthetic foot sole elements, and support the variability of foot sole element forces caused by variations in user gait in using a multiple element prosthetic foot. By using at least two separate mechanical energy harvesting paths adaption to variations in the user's gait patterns is accomplished with improved energy harvesting availability.

It is an object and intent of the present invention to improve the application of energy harvesting and control components by the prosthetic device manufacturer and application specialist by minimizing the points of contact with the existing prosthetic, not requiring any significant modification or redesign of the prosthesis, and supporting the user's need as optional installed components.

It is an object and intent of the present invention and its application methods of impulsive energy harvesting from the prosthetic foot sole to improve the cost and access to the user by using readily available component and material technologies in use in other industries. Through the advantages of competitive technology trends in component efficiency and reduced size, cost, and weight goals can be met with acceptable availability and cost.

It is an object and intent of the present invention to improve the installation process by the prosthetic application specialist and satisfy the user's aesthetic and convenience needs by having required components small enough that they can fit inside the prosthetic or natural leg physical envelope.

It is an object and intent of the present invention to minimize the impact to the user by avoiding any requirements for proximity to battery recharging equipment, and avoiding requirements for removing the prosthetic leg from use during recharging of the battery.

It is an object and intent of the present invention to provide recovered energy support for an optional RF communication function to report operating status to the user using a readily available consumer device. It is anticipated that this will address the user's needs for developing confidence in the prosthetic leg operation, allow activity planning, and increase overall confidence in prosthetic reliability.

### BRIEF DESCRIPTION OF THE DRAWINGS

FIG. 1 is an overview drawing depicting the preferred embodiment of the present invention applied to a prosthetic leg 100.

FIG. 2 is a detail drawing of the right side view of the primary embodiment of the present invention of FIG. 1 depicting major energy harvesting components for a single flexible heel element foot within a natural foot profile.

FIG. 3 is a detail drawing of the right side view of the secondary embodiment of the present invention of FIG. 1 depicting major energy harvesting components for a double flexible heel element foot, also referred to as a split foot.

FIG. 4 is a top view of the prosthetic foot embodiment FIG. 3 of the present invention depicting functional components for a split prosthetic foot.

FIG. 5 is a bottom view of the prosthetic foot embodiment FIG. 3 of the present invention depicting functional components for a split prosthetic foot.

FIG. 6 is a schematic diagram of the rectifier assembly and capacitors 45 circuitry of the present invention.

FIG. 7 is a schematic diagram of the electronic voltage regulator 46 circuit depicting hysteresis feedback control of the buck/boost regulator 60 of the present invention.

FIG. 8 depicts electrical energy flow within electronic assembly 19 of the present invention.

FIG. 9 depicts control connections to control processor 49 consisting of sensed quantities and control output signals of the electronic assembly 19 of the present invention.

FIG. 10 is a software diagram depicting the energy management operating logic of control processor 49 of the present invention providing control of the RF communication 50 and battery charging control 47 energy use.

FIG. 11 is a time sequence diagram depicting the relationship and timing of energy harvesting operation of the present invention with no electrical loads.

FIG. 12 is a time sequence diagram depicting an exemplary general form of operation of the present invention resulting from the energy harvesting characteristic depicted in FIG. 11 operating with the circuit elements depicted in FIG. 8 and FIG. 9 including the control logic of FIG. 10, and electronic circuits depicted in FIG. 6 and FIG. 7.

### DETAILED DESCRIPTION OF THE INVENTION

A preferred embodiment of the present invention APPARATUS FOR PROSTHETIC LEG VACUUM UNIT BATTERY RECHARGING is here and in figures disclosed.

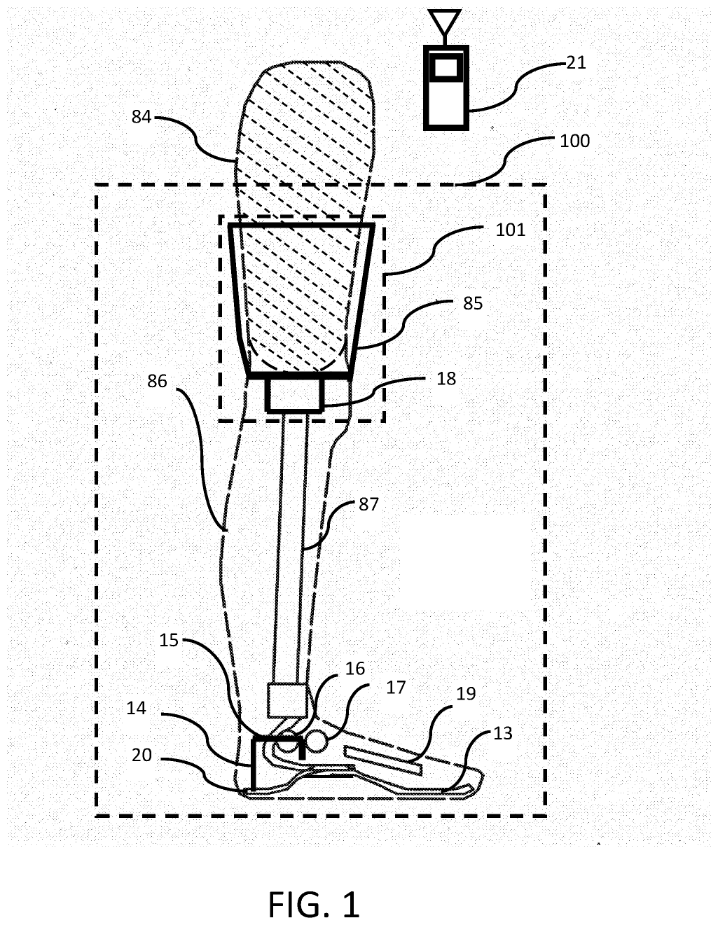

Referring to FIG. 1, prosthetic leg 100 includes residual limb vacuum socket mount assembly 101, support column 87, and attached prosthetic foot 13. The user's residual limb 84 is positioned in socket mount 85 and retained within the mount by the force resulting from the socket mount 85 inside cavity vacuum provided by battery and pump unit 18. The purpose of the present invention is to harvest mechanical energy from one of more elements of prosthetic foot 13 occurring during the use of the prosthetic leg 100, and deliver that energy to extend the operating time of the residual limb vacuum socket mount assembly 101 normally powered only by a battery within battery and pump unit 18.

Referring again to FIG. 1, flexible heel element 20 responds to the forces encountered in normal use of the prosthetic leg 100 by bending in compression as body weight is put on the leg, and extending to an unloaded position as body weight is removed. This bidirectional movement of flexible heel element 20 results in mechanical energy which can be harvested and stored, but is relatively short in duration during normal ambulation: bidirectional force impulses of tens of milliseconds duration with typical periods of one to two seconds are normally experienced.

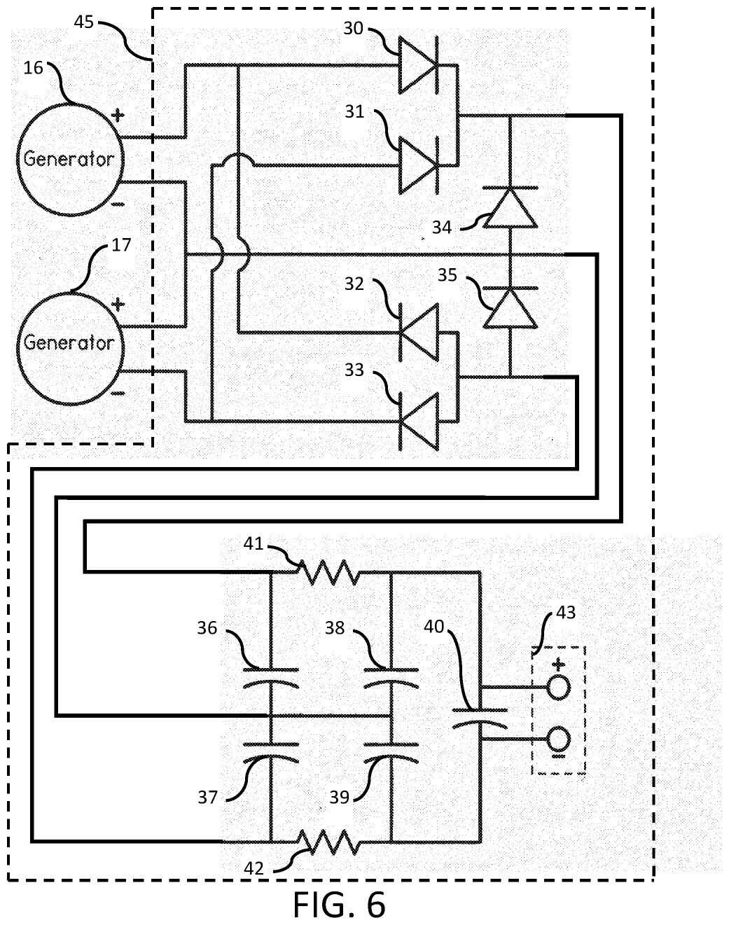

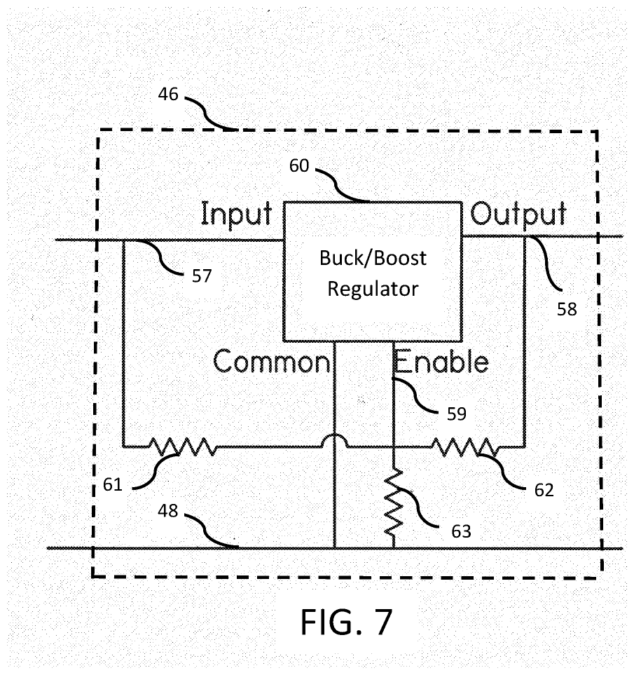

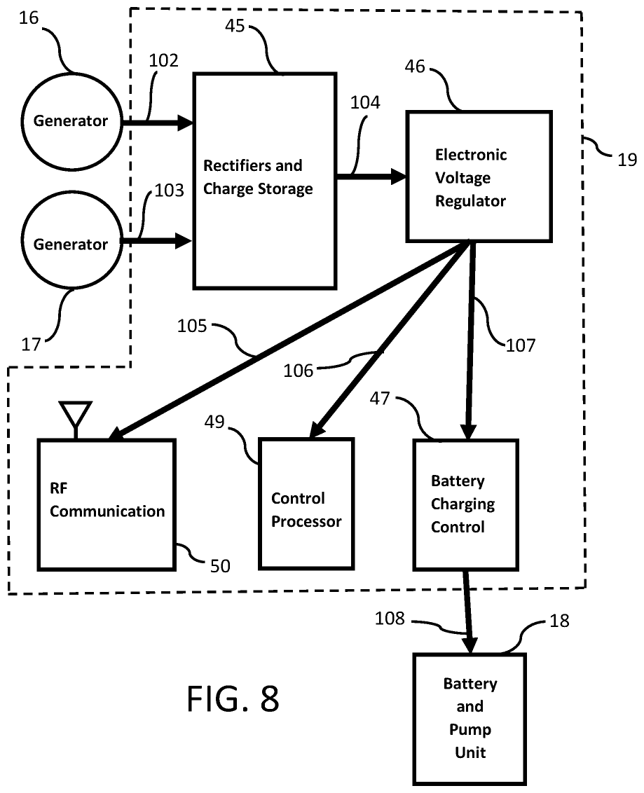

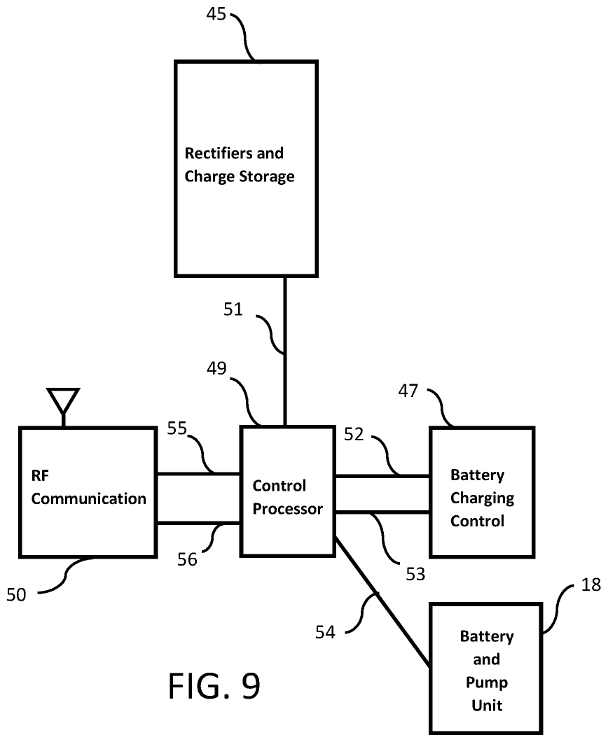

To harvest mechanical energy from prosthetic leg use, force and movement of flexible heel element 20 is coupled to generator 16 and generator 17 using force limiting link and spring 14 and drive gear 15. Generator 16 and generator 17 convert the mechanical energy to electrical energy. In the preferred embodiment of the present invention, generator 16 and generator 17 are DC generators operating with rotation in both directions. Generator 16 and generator 17 are selected to minimize losses due to operation under the intermittent mechanical conditions and the impulsive electrical current conditions. The electrical energy is stored in electronic assembly 19, and subsequently used to provide electrical energy to battery and pump unit 18. In this manner the dependency of battery and pump unit 18 on stored battery energy alone is reduced, and the total of available energy is related to the user's level of activity in ambulation with the prosthetic leg. Further details of the circuits within the electronic assembly 19 are depicted in FIG. 6, FIG. 7, FIG. 8, and FIG. 9.

Each user's prosthetic leg is a uniquely fitted device according to the physiology and needs of the user, whose amputation may be above, at, or below the knee. In each user's case the residual limb 84 portion is fitted to a surface contour conforming socket mount 85 which provides a physical interface to the prosthetic leg. It is to be appreciated that vacuum socket mount assembly 101 and battery and pump unit 18 associated with the present invention may be located in various positions along the user's prosthetic leg 100 according to the user's needs without altering the inventive concepts and principles embodied therein.

An individual user's application may or may not result in a set of components contained within the enclosed space of a three dimensional volume bounded by the natural leg boundary 86. Battery and pump unit 18 may also include external service connections for initial battery charging, adjustments and settings of the pump portion, and other service purposes according to the specific manufacturer (not shown). Battery status of battery and pump unit 18 is reported via an RF communication from electronic assembly 19 to user device 21. This allows the user to be aware and respond appropriately to prosthetic leg battery and pump unit 18 condition.

Energy is harvested from the force and deflection of prosthetic foot 13 elements, which in this depiction includes one flexible heel element 20 with an attached force limiting link and spring 14. It is to be appreciated that energy may be harvested from the force and deflection of one or more prosthetic foot 13 elements, including additional heel or forefoot elements, each equipped with force links and energy harvesting generators without altering the inventive concepts and principles embodied therein.

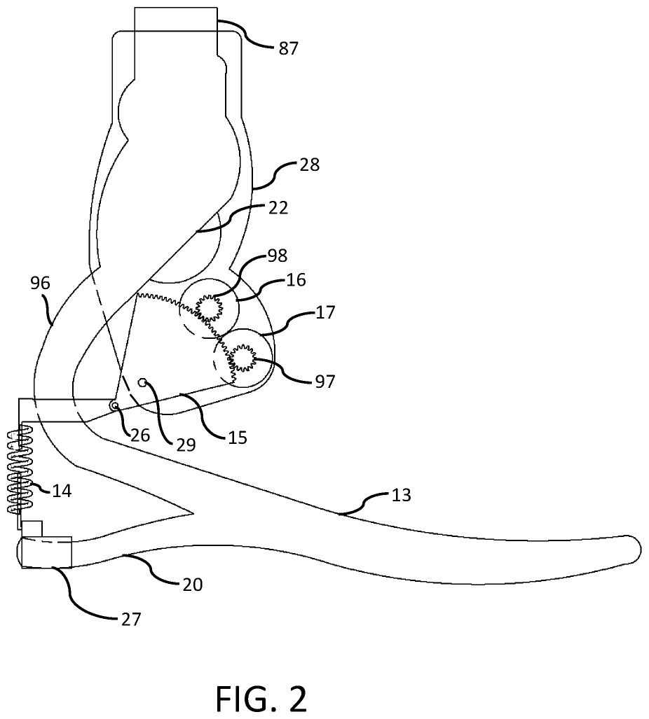

Referring to FIG. 2 of the primary embodiment, prosthetic foot ankle 22 is connected to the prosthetic foot 13 by foot structure 96, which may or may not have a flexible characteristic. Flexible heel element 20 provides significant movement in response to force on the heel. Mounting plate 28 mounted to the prosthetic foot ankle 22 provides mounting support for generator 16, generator 17, and gear pivot 29. The generator 16 and generator 17 each have a pinion gear on their shaft, pinion gear 98 and pinion gear 97, which engage the mating profile of drive gear 15. Drive gear 15 pivots on gear pivot 29. Force limiting link and spring 14 is coupled to drive gear 15 using the swivel 26. Flexible heel element 20 is coupled to the force limiting link and spring 14 using a heel attachment sleeve 27.

Energy harvesting occurs using the force and relative movement of the mechanical mechanism mounting points of the prosthetic foot ankle 22 and the heel attachment sleeve 27 on flexible heel element 20. As the user steps and uses the prosthetic leg to support their weight, force is developed on the flexible heel element 20. Through heel attachment sleeve 27, compressive force is developed on the force limiting link and spring 14, which in turn causes movement of attached drive gear 15. Movement of attached drive gear 15 causes generator 16 and generator 17 shaft rotation through the force coupling by pinion gear 98 and pinion gear 97. Force limiting link and spring 14 provides a limit on the acceleration and peak force stresses of the drive gear 15, pinion gear 98, pinion gear 97, generator 16, and generator 17.

Flexible heel element 20 of prosthetic foot 13 has a spring characteristic, such that a force causing deflection will store energy in flexible heel element 20. When the user force on the heel is removed, compressed flexible heel element 20 causes the force limiting link and spring 14 to extend, causing the drive gear 15 to pivot, which causes a reversed rotation of generator 16 and generator 17. Generator 16 and generator 17 shaft rotation results in a voltage output and current flow in their connected circuits. Reversed generator 16 and generator 17 shaft rotation results in a reversed polarity of voltage output and reversed current flow in their connected circuits.

Those skilled in the art will recognize that the response characteristics of generator 16 and generator 17 must be appropriately matched to the magnitude and dynamics of the forces applied, and to the connected electrical circuits to develop the desired energy conversion and harvested electrical energy. The force and duration applied to prosthetic foot 13 flexible elements varies significantly, particularly at the heel. Applied force to the heel is often referred to as heel strike, which results in flexible element compression. Removal of the force results in extension of the heel flexible element. Ambulation typically produces a heel strike action with a duration range of 10 milliseconds to 150 milliseconds. The action of extension is typically longer in duration.

Those skilled in the art will recognize that using a rotating electric generator to efficiently provide energy conversion of this force impulse is a technical challenge, as the conventional rotating DC electric machines with a magnetic material armature core as a rotating element in the size range of interest of from 2 to 30 Watts continuous rating have a typical mechanical time constant on the order of 50 to 100 milliseconds. This time constant is too large to effectively accomplish energy harvesting of the short duration force impulses without adding additional mechanical components. To accomplish direct energy harvesting of the relatively short force impulses present at the foot bottom surfaces, at the power level in the range of 2 to 30 Watts average continuous rating, and responsive within the time frame of the impulses requires a generator and linkage assembly with a low value of mechanical time constant.

In order to meet the mechanical time constant and electrical load requirements, multiple electric machines can be used. This also aids in meeting mounting space requirements and production of the desired generated voltages. It is to be appreciated that the electrical generating capability of each generator may be varied according to need, use of one or two generators per harvesting input may be used, and the impedance of each generator may be selected to optimize impulse energy harvesting characteristics and efficiency. The number of harvesting circuits may be varied in design and application of the present invention without altering the inventive concepts and principles embodied therein.

The coreless armature DC electric machines in the continuous load rating range of from 2 to 30 Watts applied in the present invention, generator 16 and generator 17, have typical mechanical time constants of approximately 10 milliseconds, and are exemplary of electric machine technology which make this direct coupled application feasible by being able to respond to the short duration heel strike mechanical inputs. In addition, improved magnet technology and manufacturing techniques have significantly increased the energy density and reduced armature impedance, increasing energy harvesting efficiency while reducing physical size.

Those skilled in the art will recognize that the characteristics of force limiting link and spring 14 are important in the performance and management of generator 16 and generator 17 response to heel strike force. The force limiting link and spring 14 provides both a limit to the maximum value of applied force to drive gear 15 and some energy storage to aid in shaping the duration of generator response, and increasing the harvested energy. The force limiting link and spring 14 may consist of a form of slip clutch and spring, or force limiting may be accomplished by the spring alone.

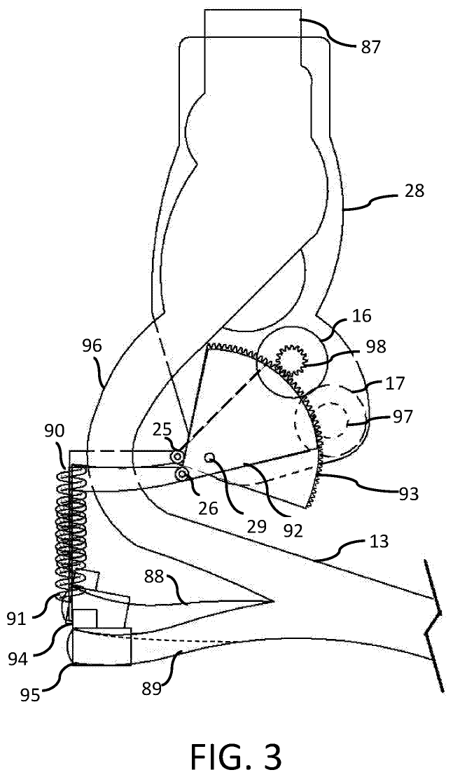

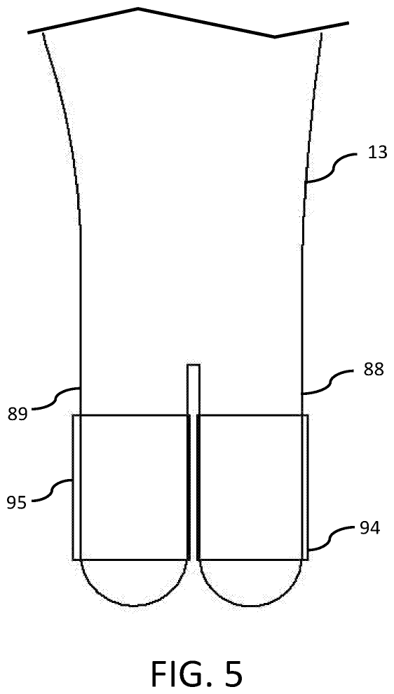

Referring to FIG. 3 and FIG. 5, the flexible heel element 20 of FIG. 2 is configured as a split heel into two flexible portions: left heel element 88 and right heel element 89. Generator 16 and generator 17, as well as left drive gear 93 and right drive gear 92 are mounted to mounting plate 28 using a similar method to that depicted in FIG. 2, with the exception that generator 17 is facing left, opposite to generator 16 facing right. Using this mounting arrangement heel element 88 and heel element 89 each drive one generator, generator 17 and generator 16 respectively.

Those skilled in the art will recognize that flexibility to vary the amount and location of energy harvesting from the foot sole can be important in meeting an individual user's needs and gait pattern. Depending on the user's gait and movement activity, the forces and deflection between the bilateral elements and other sole elements can vary significantly. By providing energy harvesting links independently connected to each bilateral foot element, the total energy harvesting can remain more consistent despite asymmetries in foot sole element forces.

In the embodiment of FIG. 3, heel element 88 and heel element 89 of the prosthetic foot 13 are equipped with separate energy harvesting components. The left heel element 88 is depicted flexing upward toward the ankle. The right heel element 89 is depicted in an unforced, lower position. On the right side of prosthetic foot 13, heel attachment sleeve 95 is connected to the right force limiting link and spring 91. Right force limiting link and spring 91 is connected to the right drive gear 92 using swivel 26. Right drive gear 92 engages a pinion gear 98 on the shaft of generator 16. On the left side of prosthetic foot 13, heel attachment sleeve 94 is connected to the left force limiting link and spring 90. Force limiting link and spring 90 is connected to the left drive gear 93 using swivel 25. Left drive gear 93 engages a pinion gear 97 on the shaft of generator 17.

Those skilled in the art will recognize that a variation of this split heel configuration is possible by connecting two generators to each flexible heel element 88 and heel element 89, as depicted for a single flexible heel element 20 in FIG. 2. This variation allows the designer to select components as needed to meet any increased energy harvesting capacity requirements.

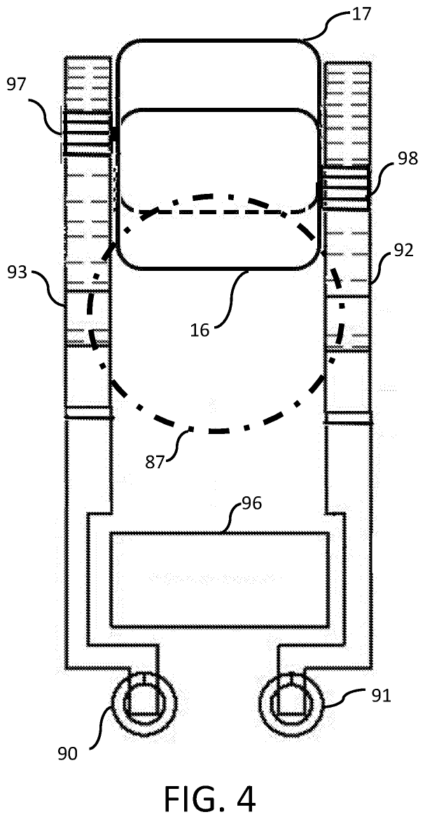

Referring to FIG. 4, the relative position of components with regard to foot structure 96 is depicted. The left force limiting link and spring 90 is connected with left drive gear 93. Left drive gear 93 engages pinion gear 97 to rotate the shaft of generator 17. Using a similar method, right force limiting link and spring 91 is connected with right drive gear 92. Right drive gear 92 engages pinion gear 98 to rotate the shaft of generator 16. Mounting plate 28 which supports generator 16 and generator 17 is not shown.

Referring to FIG. 5, heel attachment sleeve 95 and heel attachment sleeve 94 are attached to flexible right heel element 89 and left heel element 88 respectively. The attachment method of the present invention uses sleeves to meet the requirement for ease of installation and no requirement for modification of the prosthetic foot 13.

Referring to FIG. 6, the purpose of rectifier assembly and capacitors 45 circuits is to harvest the energy of the relatively short time duration, high current pulses delivered by the generator 16 and generator 17 responding to bidirectional impulsive input forces. In this exemplary application, the outputs of generator 16 and generator 17 are in phase for flexible heel element 20 compression mode and extension mode. The generator outputs are described as “in phase” when their outputs correspond as to polarity and timing with respect to the flexible element mode of compression or extension. Rectifiers 30, 31, 32, and 33 form two half bridge circuits, one for generator 16 and one for generator 17. The rectifiers 34 and 35 complete the other half of a full wave bridge in conjunction with rectifiers 30, 31, 32, and 33.

Generator 17 is connected with reverse polarity from the connection of generator 16, such that both polarities of the half wave rectifier network within rectifier assembly and capacitors 45 are driven simultaneously to charge both capacitors 36 and 37. Capacitors 36, 37, 38, 39, and 40 are all large value capacitors for charge storage, selected to support the desired energy harvesting, in this case in the range 0.005F to 0.05F. Capacitors 36 and 37 are selected for the relatively large impulse currents produced by the generators. High energy density capacitors 38 and 39 provide the majority of charge storage. Capacitor 40 is selected to provide a lowered output impedance and also provides some additional charge storage.

Resistors 41 and 42 are used to provide some limitation of the charging impulse currents from generator 16 and generator 17 to the high energy density capacitors 38 and 39. High energy density capacitors 38 and 39 are used in this circuit to provide a large capacity with relatively low physical volume. The DC output connection from rectifier assembly and capacitors 45 is present at output terminals 43.

To manage the simplicity, cost, and size of this embodiment the rectifiers in FIG. 6 are selected as conventional solid-state diodes. To increase energy harvesting efficiency, those skilled in the art will recognize that these rectifiers can be replaced by MOS transistors operated and controlled as synchronous rectifiers, or synchronous rectifiers can be placed in parallel with the rectifiers shown. This can reduce the voltage drop and conversion efficiency losses associated with rectification under most operating conditions, and additionally give the ability to control current levels.

Those skilled in the art will further recognize that energy harvesting capacity can be further increased by the addition of switching circuits configured for voltage boosting to increase generator current. By using this approach stored energy may be maximized, and some independence from input generator relative impulse timing achieved.

Those skilled in the art will further recognize that the form of the rectifier and capacitor network of rectifier assembly and capacitors 45 are intended to accommodate multiple generator inputs and increase the available output voltage with a cascade structure. The purpose of this cascade structure is to tend to balance voltages when driven by unbalanced generator sources, and asymmetrical outputs from each generator. This imbalance occurs due to the difference in waveform timing between generator outputs during the sole element compression mode compared to the output during the extension mode.

A goal of the rectifier structure and storage capacitors is to consistently harvest available energy when the generators are mechanically driven by individual flexible sole elements. As the user walks, a sequence of force impulses is created as the artificial foot flexible elements are engaged in the sequence to produce controllable leg support forces. Individual force impulses may or may not be coincident in time, and of the same polarity. To maximize energy storage, charge storage should occur, as much as possible, for each input impulse from each generator, regardless of polarity or timing. This is generally accomplished using the cascade structure and generator polarity connections shown in FIG. 6.

FIG. 2 and FIG. 3 depict the application of the present invention to one and two flexible foot elements respectively. A similar method is used to perform energy harvesting on any additional flexible foot sole elements. The designer may choose to apply two or more generators to a force link for several reasons.

First, the use of two generators connected to the rectifier assembly and capacitors 45 as depicted in FIG. 6 provides active energy harvesting output to all energy storage elements of rectifier assembly and capacitors 45. Second, the designer may choose to select the second generator to serve as a load splitting element, such that the energy harvesting load of the single force link is split between the two generators. By reducing the physical size of each generator, mounting these components within the prosthetic foot envelope may be easier to achieve.

Third, by selecting a second generator, system reliability is improved. In the event of one generator failure, energy conversion efficiency is affected and the amount of energy conversion may also be reduced. The benefit of this condition is that the energy conversion operation will still continue, and not cease as would be the case with a single generator.

Referring to FIG. 7, the purpose of electronic voltage regulator 46 is to receive the variable stored charge from output terminals 43 of rectifier assembly and capacitors 45 at regulator input 57 and common connection 48, and produce regulated DC voltage between regulator output 58 and common connection 48. The buck/boost regulator 60 converts the voltage appearing between regulator input 57 and common connection 48 to a stable voltage appearing at regulator output 58. The enable terminal 59 of buck/boost regulator 60 causes it to operate when the voltage at that terminal is above a fixed threshold value, and cease operation when the voltage is below the fixed threshold value.

The network of resistor 61, resistor 62, and resistor 63 operate with the enable terminal 59 of buck/boost regulator 60 to create a hysteresis control function, which causes buck/boost regulator 60 to start operation at a relatively high value of input voltage, and only cease operation when the input voltage decreases to a relatively low value. The value of input voltage at which the buck/boost regulator 60 starts operation is determined by the circuit designer based on the energy requirements and program execution time of the control processor 49. The purpose of this control function of the present invention is to draw stored charge from rectifier assembly and capacitors 45 only when there is sufficient energy available to provide battery charging or RF communication functions. The purpose of the buck/boost function is to provide as wide a voltage range as possible so that the available energy storage and energy use is maximized.

Those skilled in the art will recognize that for buck/boost regulator 60 a switching regulator circuit may best help meet the overall size and efficiency requirements.

Referring to FIG. 8, the electrical energy flow of the present invention is shown occurring over two conductor links 102, 103, 104, 105, 106, 107, and 108. Generator 16 and generator 17 are connected independently to a rectifier assembly and capacitors 45 using two conductor links 102 and 103, respectively. When a single heel element is used, generator 16 and generator 17 operate mechanically in parallel and are driven by the force limiting link and spring 14 and drive gear 15 as depicted in FIG. 2. The rectifier and DC charge storage 45 converts the bipolar generator outputs to a filtered DC value which varies according to the activity level of the generators.

The large capacitance provided by the rectifier assembly and capacitors 45 accumulates impulses of energy produced by the generators as electrical charge, which is conducted via link 104 to the electronic voltage regulator 46 to produce the electrical current used by the battery charging control 47 via link 107, control processor 49 via link 106, and RF communication 50 via link 105. The battery charging control 47 is connected to deliver current to the battery and pump unit 18 via link 108. When generator 16 and generator 17 are not operating mechanically in parallel, but are independent, the rectifier assembly and capacitors 45 provides a similar function to provide energy storage.

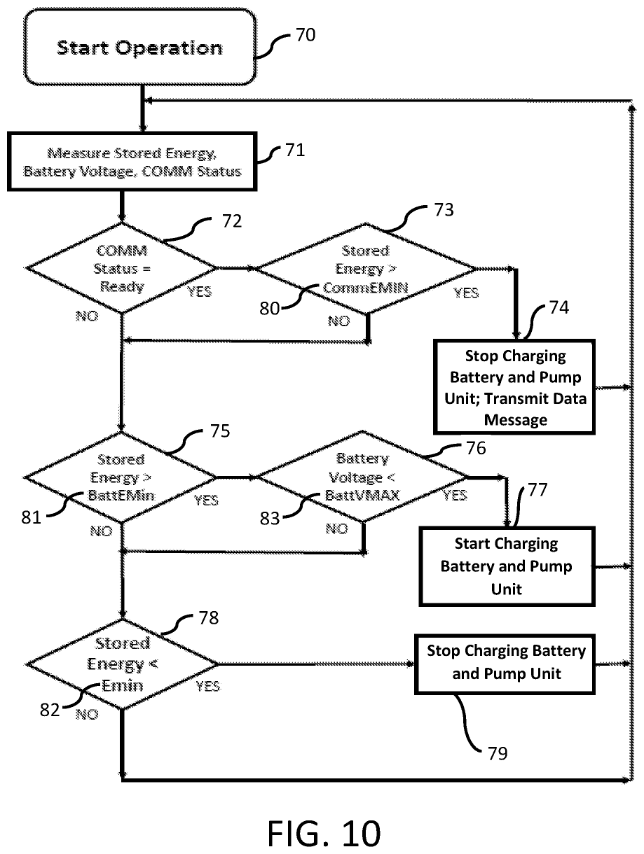

After the depletion of energy stored in rectifier assembly and capacitors 45, operation of control processor 49 starts after sufficient energy harvesting is complete and output voltage from electronic voltage regulator 46 is present. Sufficient energy harvesting is determined by the hysteresis control function within electronic voltage regulator 46. After control processor 49 starts, battery charging control 47 and RF communication 50 are controlled by software operation of the control processor 49, described in FIG. 10.

Referring again to FIG. 8, those skilled in the art will recognize that the polarity and characteristics of the two conductor links 104, 105, 106, 107, and 108 are determined by the requirements of the specific electronic circuits and components applied by the designer. In order to optimize the amount of energy harvesting, the polarity and characteristics of the two conductor links 102 and 103 connected to generator 16 and generator 17 will vary according to the characteristics of the mechanical energy harvesting links selected by the designer. For generator 16 and generator 17 operating with outputs predominantly in phase with each other, the polarity of the two conductor links 102 and 103 will be out of phase with each other. For generator 16 and generator 17 operating with outputs predominantly out of phase with each other, the polarity of the two conductor links 102 and 103 will be in phase with each other.

Application of the present invention involves matching the flexible sole element forces and deflection, force limiting link and spring 14, outputs of generator 16 and 17, rectifier assembly and capacitors 45, battery charging control 47 and battery and pump unit 18. The battery and pump voltage must be matched with appropriate component selections through all of these elements.

In the present invention, force link ratings, link gear ratio, generator internal gear train ratio, and electronic component ratings can all be selected to meet the battery and pump unit 18 requirements. By performing energy harvesting on more than one prosthetic foot sole flexible element, not only can more energy be harvested, but energy harvesting can be done in a manner to accommodate different ambulation sequences and speeds, which typically apply force to the different foot flexible elements in differing amounts and with differing durations.

A force limiting link is selected to allow no more than what can be reliably accommodated by the gear, generator shaft, and other affected mechanical components. Due to the impulsive nature of applied force to the prosthetic foot sole, peak force values are often in excess of an order of magnitude greater than the value expected for continuous energy harvesting.

Limiting peak forces to within practical ratings for mechanical components allows a reliable design to be accomplished. This includes the maximum acceleration for rotating components, such as generator 16 and generator 17. Generators selected from motors designed for high performance servo control applications typically have high acceleration ratings and shaft peak force abilities, with corresponding armature winding and commutator current ratings. In addition, it is important to note that the mechanical response of the force link and gear system can be underdamped, and somewhat oscillatory in form. This is due to the stored energy in the flexible elements and spring, link mass, and the rotating elements moment of inertia. Active energy harvesting tends to minimize the underdamped response amplitude.

A desirable characteristic of the generators is low impedance. By keeping generator impedance low, terminal voltage is kept high under active energy harvesting conditions, and efficiency is improved. This is particularly true as force/time sole inputs become more impulsive in form, and peak generator currents are increased.

Referring to FIG. 9, control processor 49 sensing and control line connections of the present invention are shown connected to battery charging control 47 and RF communication 50, the two processor controlled loads in electronic assembly 19. The control processor 49 senses the stored energy level by sensing capacitor voltage line 51 which is connected to the output terminals 43 of rectifier assembly and capacitors 45. The control processor 49 senses battery condition by sensing battery voltage through battery voltage connection line 54 from battery and pump unit 18. Battery charging control 47 is controlled by the control processor 49 using an enable line 52 and a charging control level 53. The battery charging control 47 converts the supplied regulated DC voltage received from electronic voltage regulator 46 to a battery charging current appropriate for the battery in battery and pump unit 18 and operates as directed by the control processor 49. The RF communication 50 is operated by RF communication control line 55 and data communication line 56.

Those skilled in the art will recognize that the battery charging control 47 operation can be enhanced by using the information from stored energy measurement from capacitor voltage line 51 in the rectifier assembly and capacitors 45 and battery voltage connection line 54 to set the charging current level using charging control level 53. Using this information the control processor 49 can be used to set the battery charging current as high as the stored energy available will support, the current energy harvesting rate will maintain, and the battery technology will allow.

The RF communication 50 provides wireless communication to a user device 21, such as a cell phone or separate receiver. RF communication 50 operates using the supplied regulated DC voltage with data provided by the control processor 49 using data communication line 56. The communicated data includes the sensed value from battery voltage connection line 54.

Referring to FIG. 10, operating logic in control processor 49 of the present invention is intended to accomplish allocation of the stored harvested energy in rectifier assembly and capacitors 45 according to prosthetic limb electrical circuit function needs and requirements. The operating logic of FIG. 10 in control processor 49 is used to determine when it is appropriate to operate each electrical load at the pre-determined power level that each load requires.

Control logic operation starts as depicted in FIG. 10 at action 70. The purpose of the energy management software is to operate battery charging control 47 and RF communication 50 only when adequate energy is available from the stored supply and the desired sequence of operations can be accomplished. Operations are allowed to start only when the minimum stored energy threshold for each operation exceeds the required energy for the operation, as set by parameters in the software operating in control processor 49.

The initial action in the energy management software after starting is to measure stored energy represented by rectifier module capacitor voltage as communicated via capacitor voltage line 51, battery voltage as communicated via battery voltage connection line 54, and communication status as communicated via data communication line 56 represented in action 71. Thereafter, logic test 72 is performed, which checks communication status for the readiness of RF communication 50. The RF communication 50 is ready for operation if the recorded time since the last RF communicated message was broadcast is greater than a fixed, pre-programmed time COMMPeriod, and the RF communication 50 internal circuits are ready to accept data.

If the RF communication 50 is ready, operation of the software proceeds to logic test 73. If not, operation of the software proceeds to logic test 75. COMMPeriod is intended to be chosen by the designer to be the minimum time between broadcast communications to external devices, such that the number of broadcast communications satisfies the need for sample data by the user and is not so frequent that the energy expended in communications decreases the energy available for prosthetic operation to an unacceptable level.

When logic test 73 is performed, the measured stored energy from action 71 is compared to a fixed, pre-programmed value CommEMIN 80. If the stored energy from action 71 is greater than CommEMIN 80, operation of the software proceeds to action 74. If the stored energy from action 71 is not greater than CommEMIN 80, operation of the software proceeds to logic test 75. The pre-programmed value CommEMIN 80, compared to the measured voltage of the energy storage capacitor 40, is intended to be chosen by the designer to be the minimum value sufficient to support the complete broadcast RF communication process and power needs of the RF communication 50.

When action 74 is performed, the control processor 49 operates to stop operation of the battery charging control 47 using enable line 52 and a charging control level 53. Continuing action 74, control processor 49 starts operation of the RF communication 50 to transmit a data message using RF communication control line 55 and data communication line 56. The data message transmitted consists of the value of battery voltage measured, which is used by the user to determine how much of the battery capacity remains. After action 74 is performed, software operation resumes with action 71. Action 74 is not complete until the complete communication operation is complete. RF communication 50 automatically reduces its energy consumption when the control processor 49 commanded communication operation is complete.

When logic test 75 is performed the measured stored energy from action 71 is compared to a fixed, pre-programmed value BattEMIN 81. The pre-programmed value BattEMIN 81, compared to the measured value of capacitor voltage line 51, is intended to be chosen by the designer to be the minimum value sufficient to support a significant value of battery charging current by battery charging control 47. If the measured value of capacitor voltage line 51 from action 71 is greater than BattEMIN 81, operation of the software proceeds to logic test 76. If not, operation of the software proceeds to logic test 78. When logic test 76 is performed, the measured battery voltage from action 71 is compared to a fixed, pre-programmed value BattVMAX 83.

If the measured battery voltage connection line 54 from action 71 is less than BattVMAX 83, operation of the software proceeds to action 77. If the measured battery voltage connection line 54 from action 71 is not less than BattVMAX 83 the control processor 49 operation continues with logic test 78. The pre-programmed value BattVMAX 83, compared to the measured battery voltage connection line 54 voltage of the battery and pump unit 18, is intended to be chosen by the designer to be the maximum value of battery voltage at full charge, such that any value of battery voltage that is lower is interpreted to mean that the battery will store charge if charging current is supplied. In the situation when the pump within battery and pump unit 18 is operating, the sensed battery voltage will be slightly lower than when the pump is not operating, allowing the battery charging control 47 to provide battery and pump unit 18 both charging and operating current.

When action 77 is performed, the control processor 49 operates to start charging of the battery by activating battery charging control 47 using enable line 52 and charging control level 53. After action 77 is performed, software operation resumes with action 71.

When logic test 78 is performed, the measured stored energy from action 71 is compared to a fixed, pre-programmed value EMIN 82. If the stored energy from action 71 is less than EMIN 82, operation of the software proceeds to action 79. If the stored energy from action 71 is not less than EMIN 82, software operation resumes with action 71. When action 79 is performed, the control processor 49 operates to stop charging of the battery by deactivating battery charging control 47 using enable line 52 and charging control level 53. After action 79 is performed, software operation resumes with action 71. The pre-programmed value EMIN 82, compared to the measured voltage of the energy storage capacitor 40, is chosen to be the minimum value sufficient to support a supply of battery charging current by battery charging control 47.

Operation of the software in control processor 49, once started, is intended to operate indefinitely. The software is only intended to stop operation when the regulated power provided by electronic voltage regulator 46 is at a value insufficient to operate control processor 49. Those skilled in the art will recognize that some energy may be saved by operating the control processor 49 periodically rather than continuously.

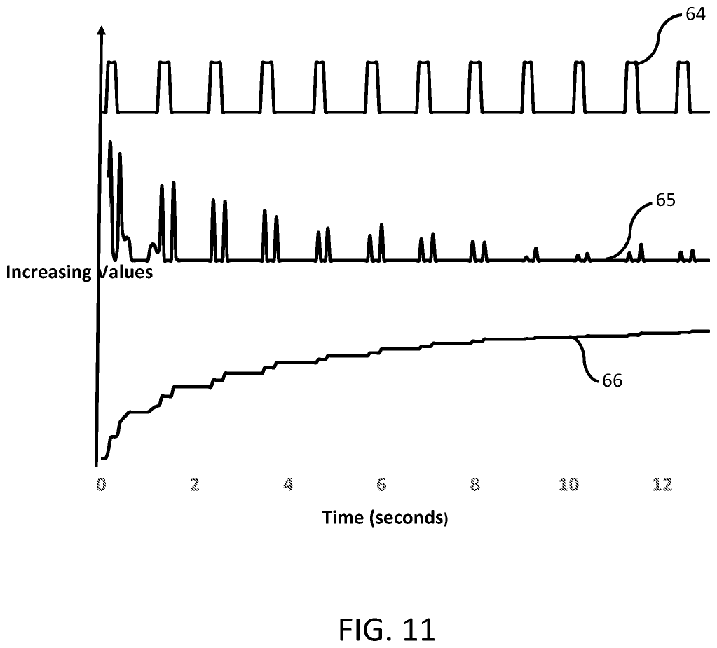

Referring to FIG. 11, the exemplary circuit operation resulting from periodic heel strike 64 occurring during prosthetic use in ambulation is shown without an electrical current being drawn from rectifier assembly and capacitors 45. The depicted energy storage voltage 66 is exemplary of the voltage present at the output terminals 43 of rectifier assembly and capacitors 45. An increase on the vertical scale in the value plotted for heel strike 64 indicates the increase in pressure on the foot heel member. An increase on the vertical scale in the value plotted for generator current 65 indicates an increase in the value of generator electrical current. An increase on the vertical scale in the value plotted for energy storage voltage 66 indicates an increase in the value of voltage on the output terminals 43 of rectifier assembly and capacitors 45, which represents the accumulated harvested energy.

As each heel strike 64 occurs there are corresponding currents flowing in the generators, shown in the plotted values of generator current 65. The occurrence of generator current 65 causes an increase in the plotted energy storage voltage 66. This exemplary form of generator current 65 shows current generated during both the heel compression mode of ambulation, as well as heel extension mode when the foot is lifted.

Those skilled in the art of electrical circuit operation will recognize the general form of the unloaded energy harvesting seen in energy storage voltage 66 as an exponential response. Those skilled in the art of electrical circuit operation will also recognize the decrease in the generator current 65 over time corresponding to the increase in energy storage voltage 66. As depicted in FIG. 11, without an electrical load on the output of rectifier assembly and capacitors 45 its voltage output will reach a maximum value dependent on the heel strike force, generator characteristics, and resulting generator operating speed.

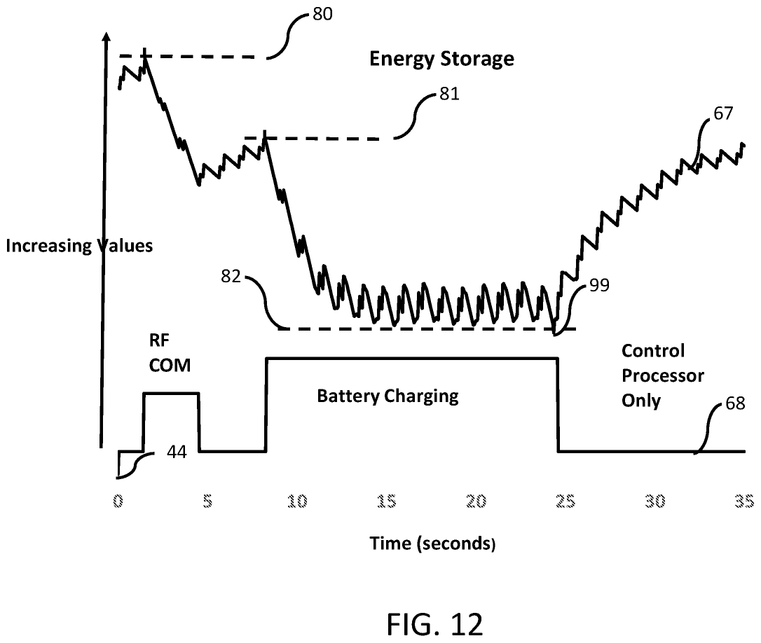

It is a general characteristic of the rectifier assembly and capacitors 45 circuit that generator current 65 will increase as energy storage voltage 66 decreases for a constant level of heel strike 64 force input. If the average rate of energy recovery exceeds the electrical load of electronic voltage regulator 46, the energy storage voltage 66 will trend towards a stable average value, as further described in FIG. 12.

Referring to FIG. 12, this exemplary sequence of operation is portrayed by the voltage appearing at the output terminals 43 of rectifier assembly and capacitors 45 which appears as voltage 67, and the load current of the electronic voltage regulator 46 appears as load current 68. Starting operation at the point 44, load current 68 is briefly zero until the control processor 49 starts after the electronic voltage regulator 46 starts operation and the load current 68 is at a relatively low level. As the control processor 49 performs the control logic operations depicted in FIG. 10, if the control logic conditions are satisfied, including voltage 67 above the reference voltage CommEMIN 80, the control processor 49 starts operation of RF communication 50. At that point load current 68 increases as RF communication 50 operates, and voltage 67 may decrease as stored energy is supplied to RF communication 50. As the control processor 49 completes communication operation RF communication 50 operation is stopped, and the load current 68 decreases to continue operation with the control processor 49 as the only load. During this period voltage 67 may increase due to continued energy harvesting.

As the control processor 49 continues performing the control logic operations depicted in FIG. 10, if the control logic conditions are satisfied, including voltage 67 above the reference voltage BattEMIN 81, the control processor 49 starts operation of battery charging control 47. At that point load current 68 increases as battery charging control 47 operates, and voltage 67 may decrease during this time as stored energy is supplied to battery charging control 47. During the period of time that more energy is being supplied to the battery charging control 47, the voltage 67 may decrease until the rate of energy harvesting increases to match the rate of energy being supplied. If insufficient energy harvesting occurs, the voltage 67 may continue to decrease, dropping below voltage EMIN 82 at point 99, at which point control processor 49 stops operation of battery charging control 47.

As battery charging control 47 operation is stopped at point 99, consequently load current 68 decreases to continue operation with the control processor 49 as the only load. If energy harvesting operation continues at a sufficient rate, voltage 67 may increase. If at any time the energy harvesting operation is at a low level or stops, voltage 67 may decrease to a voltage value where the electronic voltage regulator 46 can no longer operate, and control processor 49 will stop. Under this condition, operation of the electronic voltage regulator 46 will only resume if the voltage 67 rises to a sufficient level, as determined by the circuit in FIG. 7.

Those skilled in the art of electrical circuit operation will recognize that the operating characteristics are managed by the designer through choices of the electrical generation capacity, energy harvesting capacity, and energy storage capacity balanced against the energy consumption required by the prosthetic equipment loads.

It is to be appreciated and will be apparent to those skilled in the art that many variations in the implementation of the present invention are possible. An exemplary procedure for the determination of a specific implementation can start with the specification of the required output in terms of total energy, voltage, current, and dynamic responses. Applying a knowledge of the user's prosthetic application requirements in terms of forces and frequency of use can be used to determine the magnitude of harvestable energy. For the present invention the required component specifications can be determined by engineering calculations determining required capacities and power levels. The disposition and number of generators are variable according to the form of flexible prosthetic foot being used, and the desired level of energy harvesting. Current electronic component device technology can provide overall energy harvesting and conversion efficiency in the form of the present invention of approximately 50% or greater.

An exemplary variation of the generators includes those with integral gear train assemblies to increase generator speed. In concert with the mechanical link, drive gears, and generator selection a wide range of energy harvesting operating voltages and capacities are possible.

Having thus described in detail a preferred embodiment of the present invention APPARATUS FOR PROSTHETIC LEG VACUUM UNIT BATTERY RECHARGING, it is to be appreciated and will be apparent to those skilled in the art that many changes not exemplified in the detailed description of the invention could be made without altering the inventive concepts and principles embodied therein. It is also to be appreciated that numerous embodiments incorporating only part of the preferred embodiment are possible which do not alter, with respect to those parts, the inventive concepts and principles embodied therein.

The presented embodiments are therefore to be considered in all respects exemplary and/or illustrative and not restrictive, the scope of the invention being indicated by the appended claims, and all alternate embodiments and changes to the embodiments shown herein which come within the meaning and range of equivalency of the appended claims are therefore to be embraced therein. It is also to be appreciated that prosthetic applications using a vacuum assisted socket mounting sleeve for below, at, or above knee applications can be served by the present invention.

We claim:

1. Apparatus for providing electrical energy to a battery and a vacuum pump in a prosthetic leg having a flexible element that produces impulse forces during ambulation, comprising:
at least one link connected to said flexible element, said link receiving impulse forces produced by said flexible element and limiting said forces;
at least one generator connected to and receiving limited impulse forces from said link, said at least one generator converting limited impulse forces received from said link into electrical energy;
means for storing electrical energy, said means for storing electrical energy being electrically connected to said at least one generator and having at least one energy storage capacitor, said means for storing electrical energy rectifying electrical energy received from said at least one generator and storing rectified electrical energy in said energy storage capacitor;
a voltage regulator electrically connected to said means for storing electrical energy; and,
a battery charge control electrically connected to said voltage regulator and to said battery and vacuum pump.

2. The apparatus according to claim 1 wherein said at least one generator has a rotor, said rotor rotating bidirectionally in response to impulse forces received from said link and generating electricity when rotating in either direction.

3. The apparatus according to claim 2 wherein said at least one generator is a coreless DC generator.

4. The apparatus according to claim 3 further comprising a control processor, said control processor being electrically connected to said voltage regulator and being in electronic communication with said means for storing electrical energy, said battery charge control, said battery and said vacuum pump.

5. The apparatus according to claim 4 wherein said apparatus has a first and a second generator, said first and second generators producing asymmetrical electrical energy outputs, and wherein said means for storing electrical energy includes a rectifier circuit, said rectifier circuit being connected to receive and respond to said asymmetrical electrical energy outputs of said first and said second generators.

6. The apparatus according to claim 5 wherein said rectifier circuit is of the active switching type.

7. The apparatus according to claim 4 wherein said at least one generator delivers between two and thirty Watts of power and has a mechanical time constant of less than fifty milliseconds.

8. The apparatus according to claim 4 wherein said force limiting link includes a spring.

9. The apparatus according to claim 4 wherein said flexible element has a first flexible portion and a second flexible portion, each of said first and said second flexible portions being connected to said at least one generator.

10. The apparatus according to claim 4 wherein said battery is charged and said vacuum pump is operated simultaneously using electrical energy provided directly from said means for storing electrical energy through said battery charge control.

11. The apparatus according to claim 4 further comprising an RF communication device, said RF communication device being in electrical communication with said voltage regulator and in electronic communication with said control processor, said RF communication device broadcasting RF signals representing the status of said battery and said means for storing electrical energy.

12. The apparatus according to claim 4 wherein said prosthetic leg includes a foot portion having a heel and a forefoot and wherein said flexible element is in said heel.

13. The apparatus according to claim 12 wherein said flexible element has a first flexible portion and a second flexible portion, each of said first and said second flexible portions being connected to said at least one force limiting link.

14. The apparatus according to claim 12 wherein said flexible element has a flexible portion in said heel and a flexible portion in said forefoot, said generator receiving limited impulse forces from each of said heel flexible portion and said forefoot flexible portion through said at least one force limiting link.

15. The apparatus according to claim 12 wherein said control processor, said means for storing electrical energy, said voltage regulator, and said battery charge control comprise an electronic assembly, said electronic assembly being disposed in said foot portion of said apparatus.

16. The apparatus according to claim 15 further comprising an RF communication device disposed in said electronic assembly, said RF communication device broadcasting RF signals representative of the status of said battery and said means for storing electrical energy.

17. Apparatus for providing electrical energy to a battery and a vacuum pump in a prosthetic leg having a flexible element that produces impulse forces during ambulation comprising:
a first and a second electrical generator, each of said first and said second electrical generators having a rotatable element capable of bidirectional rotation, each of said generators producing electrical energy when its rotatable element rotates in response to applied flexible element force;
means for mechanically linking each of said first and said second electrical generators to said flexible element, said means for linking receiving and responding to impulse forces produced by said flexible element during ambulation, limiting said impulse forces and causing the rotatable element of each of said first and said second generators to rotate bidirectionally in response to applied flexible element force;
means for storing electrical energy, said means for storing electrical energy being electrically connected to said generator and having at least one energy storage capacitor responsive to the impulses of energy produced by said generators; and
a battery charge control electrically connected to said means for storing electrical energy, to said battery and to said vacuum pump.

18. The apparatus according to claim 17 wherein said first and said second electrical generators are coreless DC generators and wherein said apparatus incudes means for rectifying the electrical energy produced by each of said first and said second electrical generators, said means for rectifying delivered rectified electrical energy from said first and said second electrical generators to said at least one energy storage capacitor.

19. The apparatus according to claim 18 wherein said flexible element of said prosthetic leg has a first portion and a second portion, wherein said means for mechanically linking includes a first mechanical link and a second mechanical link, said first mechanical link being connected to said first portion of said flexible element and to at least one of said first and said second generators, said second mechanical link being connected to said second portion of said flexible element and being connected to at least one of said first and said second generators.

20. The apparatus according to claim 19 further comprising a voltage regulator and a control processor, said voltage regulator being electrically connected to said means for storing electrical energy and to said battery charge control and said control processor being in electronic communication with said means for storing electrical energy, said battery charge control, said battery and said pump.
